SEQUENCE LISTING (4 sequences)

SEQ ID NO: 1 - protein, 10 aa, Artificial Sequence
  Synthetic peptide
GMMCVTHCNG

SEQ ID NO: 2 - protein, 10 aa, Artificial Sequence
  Synthetic peptide
GMMCVSHCNG

SEQ ID NO: 3 - protein, 10 aa, Artificial Sequence
  Synthetic peptide
CMMNTSCMVL

SEQ ID NO: 4 - protein, 10 aa, Artificial Sequence
  Synthetic Peptide
CMMNTSCMVI

(19) United States
(12) Patent Application Publication
(10) Pub. No.: US 2021/0395311 A1
(43) Pub. Date: Dec. 23, 2021

(54) CYCLIC PEPTIDE DIMERS

(71) Applicant: New York University, New York, NY (US)

(72) Inventors: George LIPKIN, New York, NY (US); Martin ROSENBERG, Woodland Hills, CA (US)

(21) Appl. No.: 17/421,360

(22) PCT Filed: Jan. 14, 2020
(86) PCT No.: PCT/US20/13564

§ 371 Date: Jul. 7, 2021

Related U.S. Application Data
(60) Provisional application No. 62/792,139, filed on Jan. 14, 2019.

(51) Int. Cl. C07K 7/64; A61P 35/00

(52) U.S. Cl. CPC C07K 7/64; A61K 38/00; A61P 35/00

(57) ABSTRACT

Provided are cyclic peptide dimers. The cyclic peptide dimer include a first cyclic peptide attached to a second cyclic peptide. The present disclosure also provides compositions containing the cyclic peptide dimers. Also provided are methods of using the cyclic peptide dimers and/or compositions thereof to inhibit the growth of malignant cells in, for example, tumors.

### CROSS REFERENCE TO RELATED APPLICATIONS

This application claims priority to U.S. Provisional Application No. 62/792,139, filed on Jan. 14, 2019, the disclosure of which is incorporated herein by reference.

### FIELD OF THE DISCLOSURE

The disclosure relates to peptide-based cancer inhibitors.

### BACKGROUND OF THE DISCLOSURE

Contact inhibition is a property that distinguishes normal cells from malignant cells. There is a continuing need in the field of cancer therapy for the identification and development of novel molecules relating to contact inhibition.

### SUMMARY OF THE DISCLOSURE

The present disclosure provides cyclic peptide dimers that inhibit (e.g., contact inhibit) malignant cell growth (e.g., have anti-tumor activity). Also provided are methods of using cyclic peptide dimers.

In an aspect, the present disclosure provides cyclic peptide dimers comprising a first cyclic peptide attached (e.g., covalently bonded) via an amino acid residue of the first cyclic peptide to an amino acid residue of a second cyclic peptide. The first cyclic peptide and/or the second cyclic peptide may be N to C cyclized or cyclized by other means and/or methods known in the art.

In an aspect, the present disclosure provides pharmaceutical compositions comprising cyclic peptide dimers, biologically active fragments of the peptide(s) of the cyclic peptide dimers, and/or analogs thereof, and the like.

In an aspect, the present disclosure provides methods to inhibit the growth of malignant cells (e.g., methods for the treatment of tumors) in mammals. The methods of the present disclosure may be particularly suitable for solid tumors.

### BRIEF DESCRIPTION OF THE FIGURES

For a fuller understanding of the nature and objects of the disclosure, reference should be made to the following detailed description taken in conjunction with the accompanying figures.

FIG. 1 shows gel electrophoresis of the fractions derived under reducing conditions from the HPLC separation of the MCX cartridge eluates. The H₂O, ACN, Prop and THF fractions from the HPLC were bioassayed. Only the Prop fraction had CIF activity. All fractions were electrophoresed using a 10-20% polyacrylamide gradient and stained with SYPRO. The running buffer contained the reducing agent mercaptoethanol. Only the Prop fraction stained; the other fractions H₂O, ACN, and THF did not stain indicating absence of CIF. The Prop fraction was positive for CIF activity and had only a single band. The stained band was cut out and analyzed in a mass spectrometer to determine the molecular weight and amino acid composition and sequence.

FIG. 2 shows the molecular weight of CIF. Mass Spectrogram from electrophoresis of the single band of the CIF positive pooled propanol fractions derived from SFAMCM. This pooled fraction was the only one positive for CIF activity and had a single band on the electrophoretic gel, indicating a pure substance. The mass spectrogram had peaks at 1033.5, 1048.1, 1062.1, 1077.1 and 1093.1. The molecular weight differences are approximately 16, indicating the addition of 0-4 oxygens. The apparent molecular weight of CIF, the unoxygenated molecule is approximately 1033.5.

FIG. 3 shows amino acid composition and sequence of CIF peptide. Data obtained from the single band of the propanol fraction derived from electrophoresis of the CIF bioassay positive HPLC fraction showed that the amino acid composition and sequence of CIF is: Gly-Met-Met-Cys-Val-Thr-His-Cys-Asn-Gly (SEQ ID NO:1). Further analysis of this data led to the discovery that the CIF molecule is cyclic.

FIG. 4 shows the cyclic structure of the CIF molecule. The difference between the calculated MW of the CIF peptide and its actual MW by mass spectrometry is 18 Da, the MW of H₂O. A molecule of H₂O has been removed from the linear peptide to produce an additional amide bond resulting in a head-to-tail cyclic peptide.

FIG. 5 shows the existence of a CIF dimer. Mass Spectrogram of the synthetic CIF monomer revealed a large peak at 1034.66 Da, the expected molecular weight of cyclic CIF, but also, surprisingly a small peak at 2066.8 Da. The molecular weight of the small peak is approximately two times 1034.66, suggesting for the first time that CIF can exist as a dimer.

FIG. 6 shows a mass spectrogram of the synthesized dimer. The resulting preparation was a mixture of the dimer and the monomer because much of the monomer remained undimerized. It is the peak at 1034 Da. The dimer is the peak at 2068 Da. The monomer was then separated from the dimer and each was bioassayed. The monomer was CIF negative; the dimer was strongly positive. The biologically active CIF molecule is a dimer consisting of two identical cyclic monomers.

FIG. 7 shows an electrophoresis of the bioassay positive propanol fraction under non-reducing conditions. The procedure was performed as described in the section on electrophoresis FIG. 1, omitting the mercaptoethanol. The non-reduced gel demonstrates that CIF exists as both a monomer and a dimer.

FIG. 8 shows an Axima MALDI-TOF MS spectra of the propanol fraction.

FIG. 9 shows an Axima MALDI-TOF MS spectra of the oxidized methionine series. The masses are 1030.1 Da, 1050.8 Da, 1060.2 Da, 1066.5 Da, 1075.3 Da, 1082.6 Da, 1088.7 Da, 1098.4 Da, 1114.7 Da, and 1127 Da.

FIG. 10 shows a Qstar MALDI-TOF MS Spectra of the propanol fraction.

FIG. 11 shows a Qstar MALDI-TOF MS Spectra of the 1098 ion.

FIG. 12 shows an annotated MALDI-TOF MS/MS sequence spectra of the 1098 Ion.

FIG. 13 shows structural information for peptide A. The sequences listed are SEQ ID NO:1, which are head to tail cyclized and attached via a disulfide bond.

FIG. 14 shows a MALDI-TOF mass spectrum for peptide A. The mass spectrum shows a peak at 2068.59.

FIG. 15 shows structural information for peptide B. The sequences listed are SEQ ID NO:1, which are head to tail cyclized and attached via a disulfide bond.

FIG. 16 shows a MALDI-TOF mass spectrum for peptide B. The mass spectrum shows a peak at 2067.9.

FIG. 17 shows structural information for peptide C. The sequences listed are SEQ ID NO:1, which are head to tail cyclized and attached via a disulfide bond.

FIG. 18 shows a MALDI-TOF mass spectrum for peptide C. The mass spectrum shows a peak at 2068.7.

FIG. 19 shows structural information for peptide D. The sequences listed are SEQ ID NO:1, which are head to tail cyclized and attached via a disulfide bond, where one Cys residue on each sequence is acetamidomethyl (Acm) protected.

FIG. 20 shows a MALDI-TOF mass spectrum for peptide D. The mass spectrum shows a peak at 2210.33.

FIG. 21 shows structural information for peptide E. The sequences listed are SEQ ID NO:1, which are head to tail cyclized and attached via a disulfide bond, where one Cys residue on each sequence is acetamidomethyl (Acm) protected.

FIG. 22 shows a MALDI-TOF mass spectrum for peptide E. The mass spectrum shows a peak at 2210.97.

FIG. 23 shows structural information for peptide F. The sequences listed are SEQ ID NO:1, which are head to tail cyclized and attached via a disulfide bond, where one Cys residue on each sequence is acetamidomethyl (Acm) protected.

FIG. 24 shows a MALDI-TOF mass spectrum for peptide F. The mass spectrum shows a peak at 2206.2.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

All ranges provided herein include all values that fall within the ranges to the tenth decimal place, unless indicated otherwise.

As used herein, the term “isolated” means the referenced material is removed from its native environment (e.g., a cell). Thus, an isolated material (e.g., a peptide, such as, for example, a cyclic peptide or cyclic peptide dimer) can be free of one or more or all cellular components (i.e., components of the cells in which the native material occurs naturally (e.g., cytoplasmic or membrane component, and the like)). An isolated material may be, but need not be, purified.

As used herein, the terms “purified” or “pure” refer to material that has been isolated under conditions that reduce or eliminate the presence of unrelated materials (i.e., contaminants, including native materials from which the material is obtained). A purified protein/peptide is preferably substantially free of other proteins/peptides or nucleic acids with which it is associated in a cell. A purified nucleic acid molecule is preferably substantially free of proteins/peptides or other unrelated nucleic acid molecules with which it can be found within a cell.

As used herein, the term “substantially free” is used in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

As used herein, the term “anti-tumor” activity refers to any reduction in tumor mass or tumor burden after administration of the peptides or compositions pursuant to the present disclosure.

Amino acid residues are abbreviated herein as follows: phenylalanine is Phe or F; leucine is Leu or L; isoleucine is Ile or I; methionine is Met or M; valine is Val or V; serine is Ser or S; proline is Pro or P; threonine is Thr or T; alanine is Ala or A; tyrosine is Tyr or Y; histidine is His or H; glutamine is Gln or Q; asparagine is Asn or N; lysine is Lys or K; aspartic acid is Asp or D; glutamic acid is Glu or E; cysteine is Cys or C; tryptophan is Trp or W; arginine is Arg or R; and glycine is Gly or G. Additionally, unless otherwise noted, all peptides/proteins are written in the N to C direction. Further, N to C cyclized refers to head to tail cyclization.

The present disclosure provides cyclic peptide dimers that inhibit (e.g., contact inhibit) malignant cell growth (e.g., have anti-tumor activity). Also provided are methods of using cyclic peptide dimers.

In an aspect, the present disclosure provides cyclic peptide dimers comprising a first cyclic peptide attached (e.g., covalently bonded) via an amino acid residue (e.g., the sidechain of an amino acid residue, such as, for example, through the sulfur atom of a cysteine) of the first cyclic peptide to an amino acid residue of a second cyclic peptide. The first cyclic peptide and/or the second cyclic peptide may be N to C cyclized or cyclized by other means and/or methods known in the art.

Various peptides are suitable for the first and second cyclic peptide. The first and second cyclic peptides may be the same or different. The two cyclic peptides may be covalently linked, such as, for example, through a disulfide bond (e.g., one or more disulfide bonds) between cysteine residues of each cyclized peptide (e.g., a disulfide bond between a cysteine residue of the first cyclic peptide and a cysteine residue of the second cyclic peptide, or a first disulfide bond between a first cysteine residue of the first cyclic peptide and a first cysteine residue of the second cyclic peptide and a second disulfide bond between a second cysteine residue of the first cyclic peptide and a second cysteine residue of the second cyclic peptide, and the like).

In an example, a cyclic peptide of the present disclosure has the following amino acid sequence: Gly-Met-Met-Cys-Val-Thr-His-Cys-Asn-Gly (SEQ ID NO:1), where the peptide is N to C cyclized. Linear peptides of the present disclosure may be useful as precursors to the cyclic peptides. In various examples, one or more of the methionine residues are oxidized methionine residues. In various examples, one or more of the amino acid residues are sidechain protected (e.g., Cys is trityl-protected or acetamidomethyl-protected, Thr is t-butyl-protected, His is trityl-protected, and/or Asn is trityl-protected, or the like).

In another example, a cyclic peptide of the present disclosure has a sequence chosen from Gly-Met-Met-Cys-Val-Ser-His-Cys-Asn-Gly (SEQ ID NO:2), Cys-Met-Met-Asn -Thr-Ser-Cys-Met-Val-Leu (SEQ ID NO:3), and Cys-Met-Met-Asn-Thr-Ser-Cys-Met-Val -Ile (SEQ ID NO:4), and the like, where the peptide is N to C cyclized. In various examples, one or more of the methionine residues are oxidized methionine residues. In various examples, one or more of the amino acid residues are sidechain protected (e.g., Cys is trityl-protected or acetamidomethyl-protected, Thr is t-butyl-protected, His is trityl-protected, and/or Asn is trityl-protected).

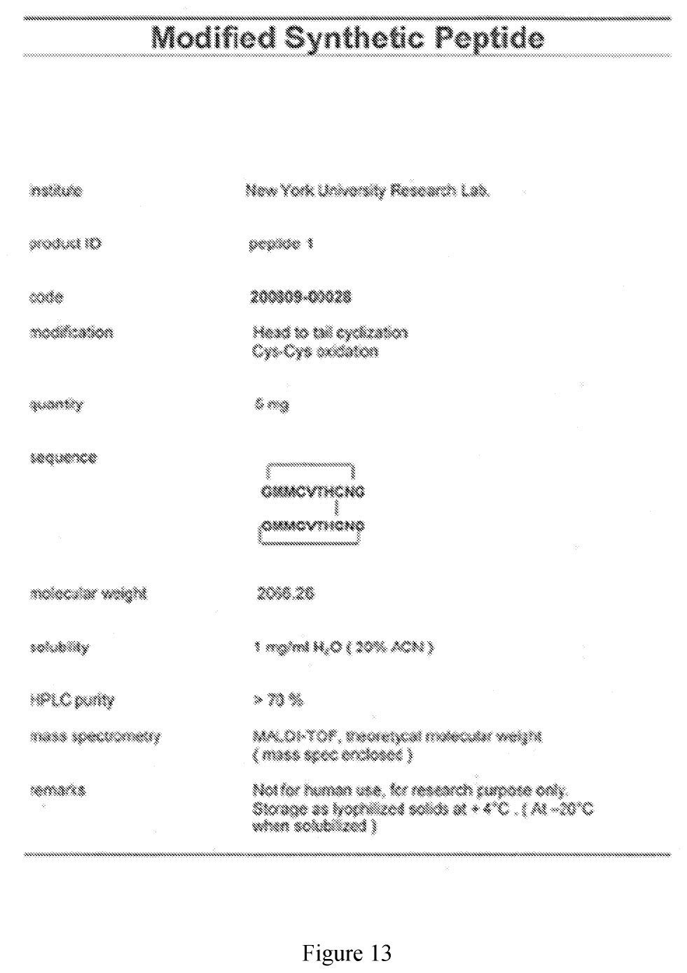

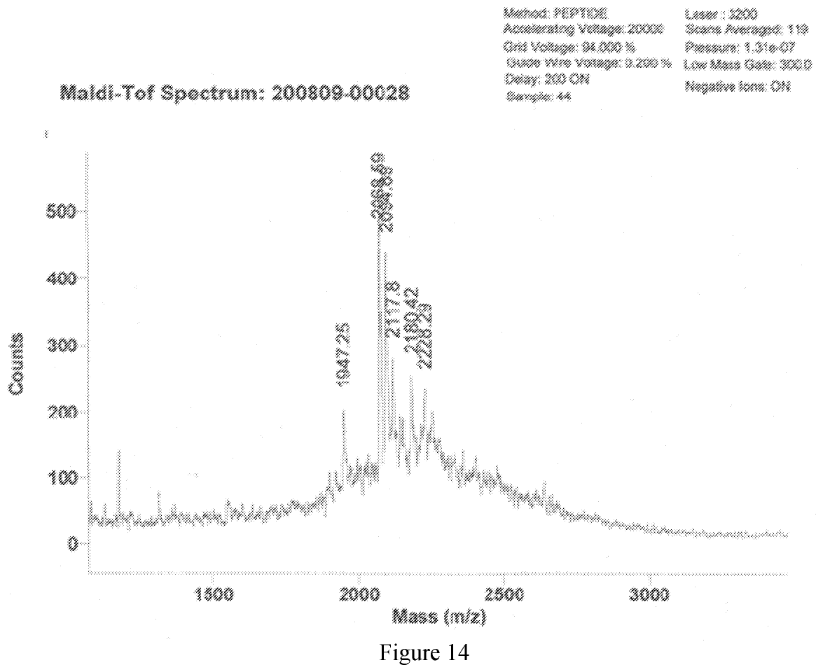

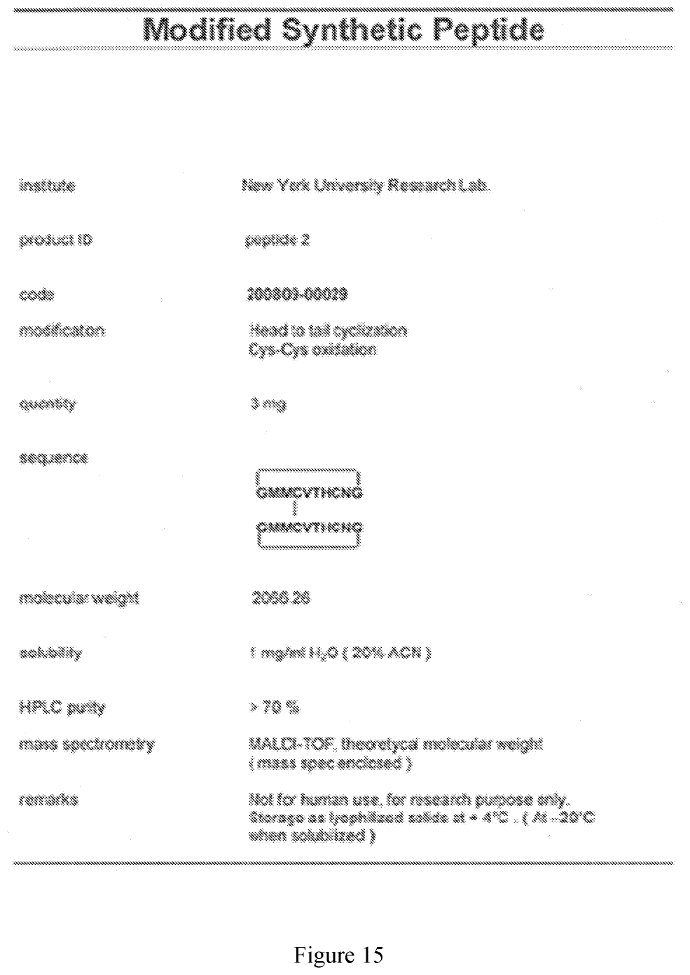

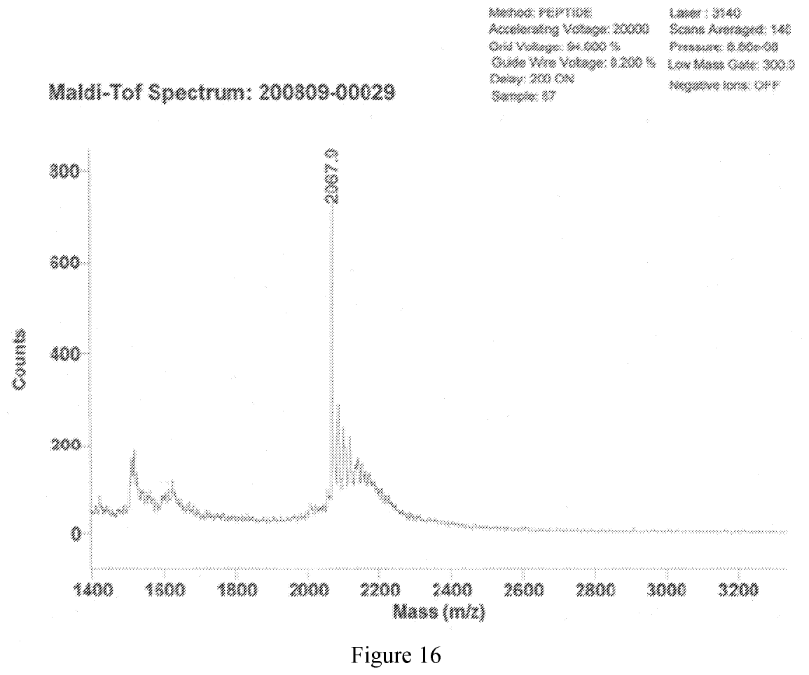

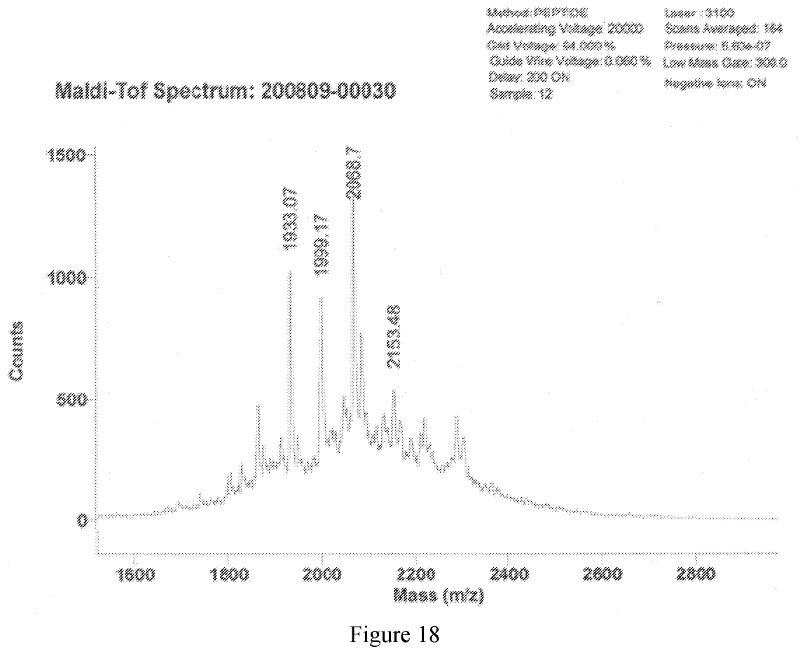

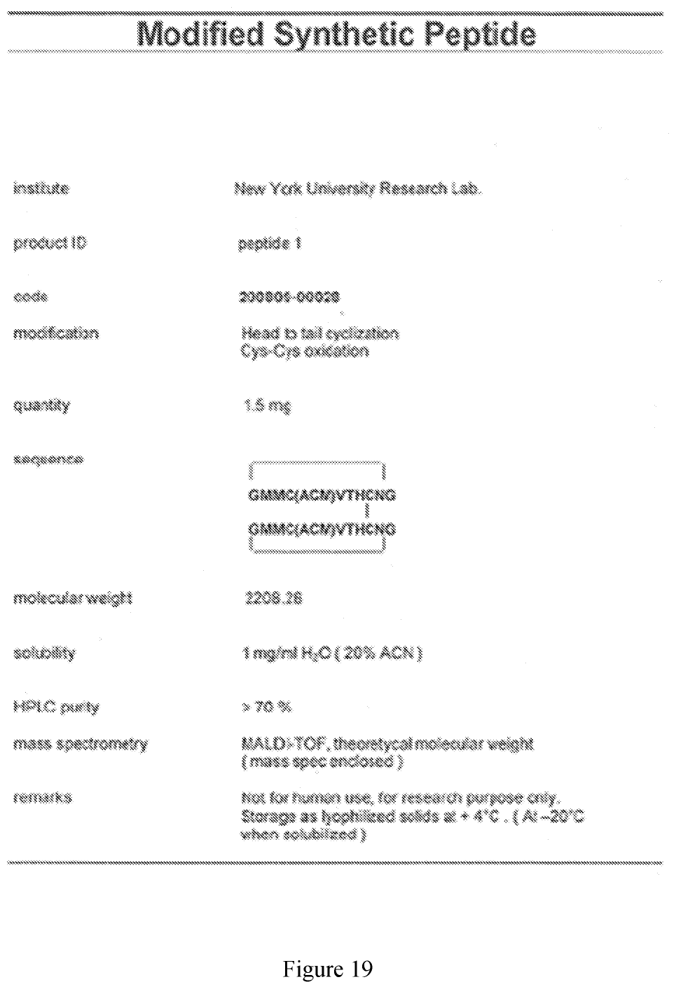

In various examples, a cyclic peptide dimer of the present disclosure has several isomers (e.g., isoforms) and the like. As an illustrative example, isomers of the cyclic peptide dimer comprising a peptide having the sequence Gly-Met-Met-Cys-Val-Thr-His-Cys-Asn -Gly (SEQ ID NO:1) include: 1) a first cyclic peptide having the sequence Gly-Met-Met-Cys -Val-Thr-His-Cys-Asn-Gly (SEQ ID NO:1), where the Cys at position 8 forms a disulfide bond with the Cys residue at position 8 of a second cyclized peptide having the sequence Gly-Met-Met-Cys-Val-Thr-His-Cys-Asn-Gly (SEQ ID NO:1) (peptide dimer A, shown in FIGS. 13 and 14); 2) a first cyclic peptide having the sequence Gly-Met-Met-Cys-Val-Thr-His -Cys-Asn-Gly (SEQ ID NO:1), where the Cys at position 4 forms a disulfide bond with the Cys residue at position 4 of a second cyclized peptide having the sequence Gly-Met-Met-Cys -Val-Thr-His-Cys-Asn-Gly (SEQ ID NO:1) (peptide dimer B, shown in FIGS. 15 and 16); 3) a first cyclic peptide having the sequence Gly-Met-Met-Cys-Val-Thr-His-Cys-Asn-Gly (SEQ ID NO:1), where the Cys at position 8 forms a disulfide bond with the Cys residue at position 4 of a second cyclized peptide having the sequence Gly-Met-Met-Cys-Val-Thr-His -Cys-Asn-Gly (SEQ ID NO:1) (peptide dimer C, shown in FIGS. 18 and 19); 4) a first cyclic peptide having the sequence Gly-Met-Met-Cys-Val-Thr-His-Cys-Asn-Gly (SEQ ID NO:1), where the Cys at position 4 forms a disulfide bond with the Cys residue at position 4 of a second cyclized peptide having the sequence Gly-Met-Met-Cys-Val-Thr-His-Cys-Asn-Gly (SEQ ID NO:1) and where the Cys at position 8 of the first cyclic peptide forms a disulfide bond with the Cys residue at position 8 of the second cyclized peptide; and 5) a first cyclic peptide having the sequence Gly-Met-Met-Cys-Val-Thr-His-Cys-Asn-Gly (SEQ ID NO:1), where the Cys at position 4 forms a disulfide bond with the Cys residue at position 8 of a second cyclized peptide having the sequence Gly-Met-Met-Cys-Val-Thr-His-Cys-Asn-Gly (SEQ ID NO:1) and where the Cys at position 8 of the first cyclic peptide forms a disulfide bond with the Cys residue at position 4 of the second cyclized peptide. Similar isomers (e.g., isoforms) exist for cyclic peptide dimers comprising peptides having the following sequences: Gly-Met-Met-Cys-Val-Ser-His-Cys-Asn-Gly (SEQ ID NO:2), Cys-Met -Met-Asn-Thr-Ser-Cys-Met-Val-Leu (SEQ ID NO:3), or Cys-Met-Met-Asn-Thr-Ser-Cys -Met-Val-Ile (SEQ ID NO:4), where the selected peptides are N to C cyclized.

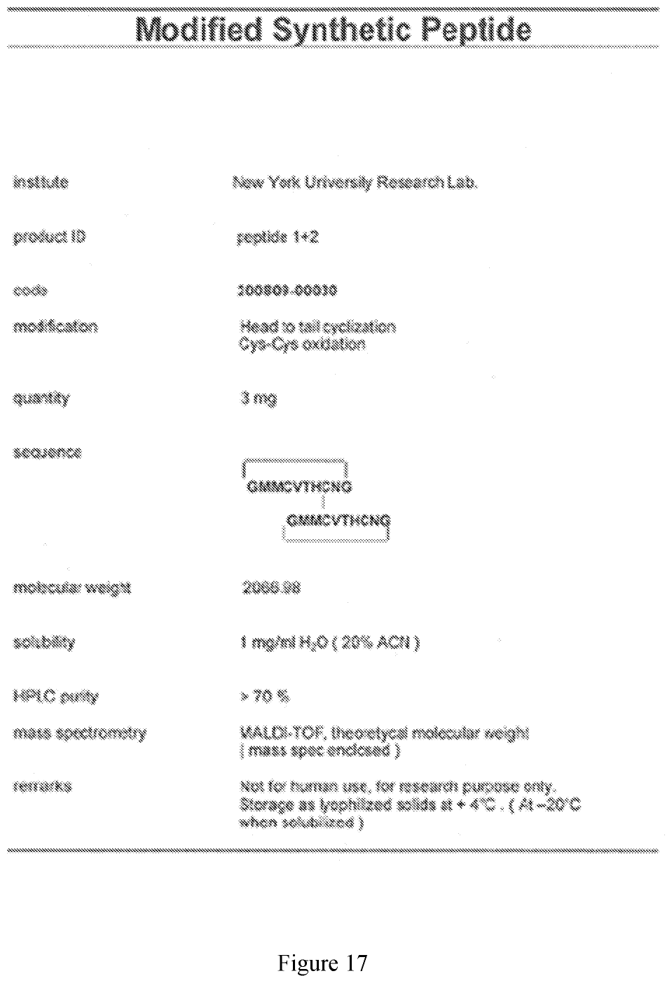

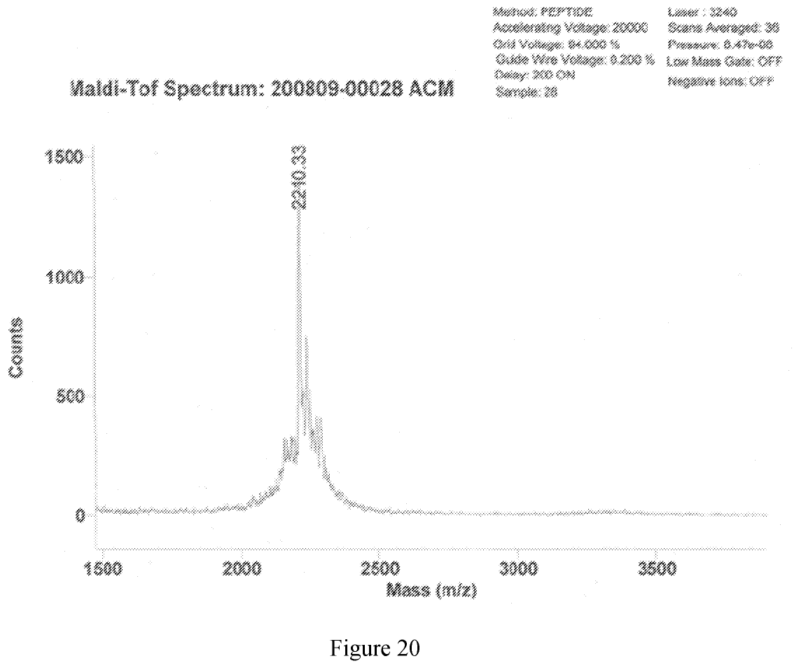

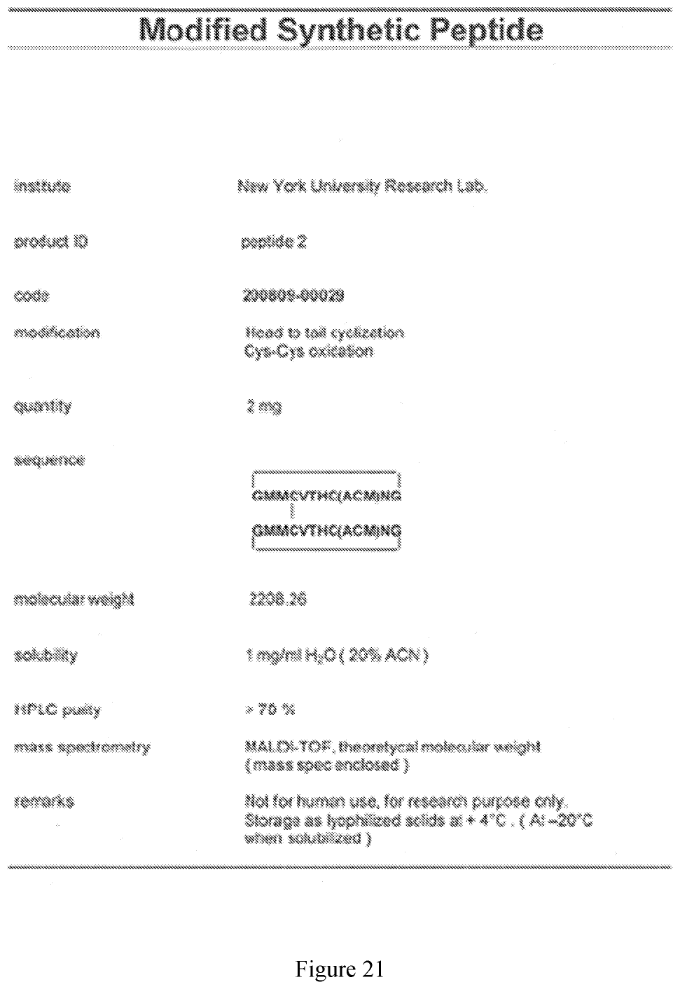

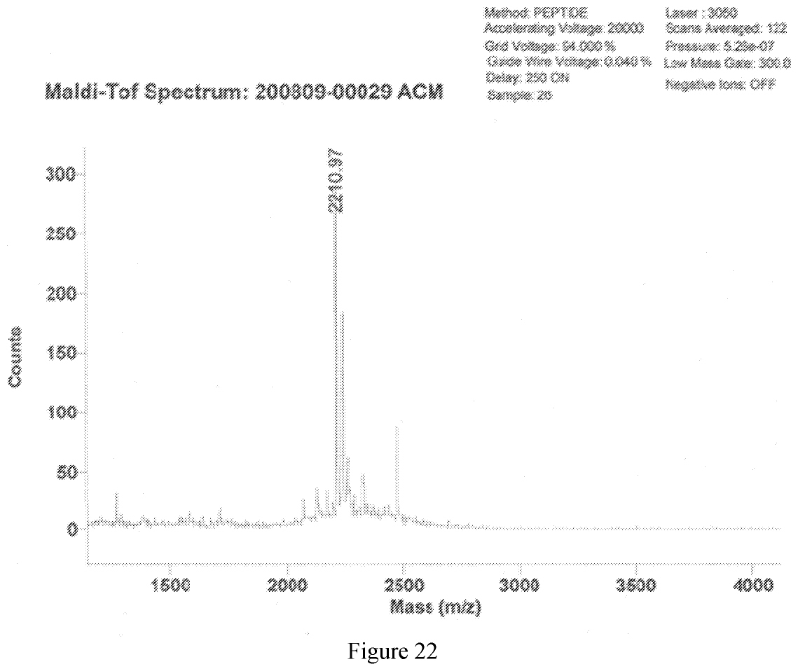

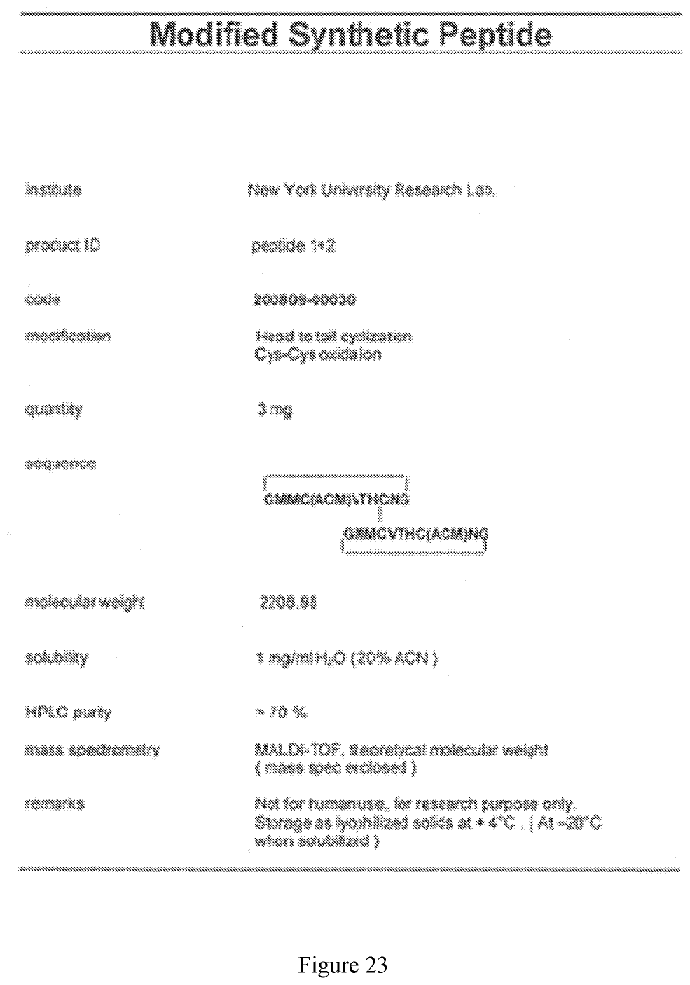

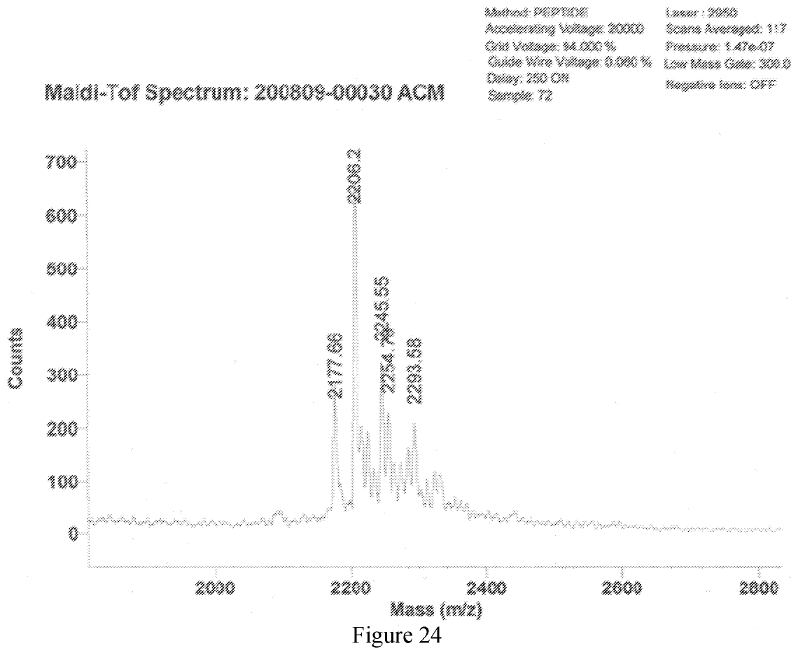

Examples of isomers of cyclic peptide dimers of the present disclosure are shown in FIGS. 13-18, which depict structural information and mass spectrometry data for peptides A, B, and C. FIGS. 19-24 depict structural information and mass spectrometry data for peptides D, E, and F, which did not have activity like the peptides A, B and C.

In various examples, cyclic peptides of the present disclosure include biologically active fragments and analogs of Gly-Met-Met-Cys-Val-Thr-His-Cys-Asn-Gly (SEQ ID NO:1), Gly-Met-Met-Cys-Val-Ser-His-Cys-Asn-Gly (SEQ ID NO:2), Cys-Met-Met -Asn-Thr-Ser-Cys-Met-Val-Leu (SEQ ID NO:3), and/or Cys-Met-Met-Asn-Thr-Ser-Cys-Met -Val-Ile (SEQ ID NO:4). “Biologically active fragments” of the cyclic peptides of the present disclosure are those with less than the original number of amino acid residues of the parent compound. A parent compound may be a peptide from which the biologically active fragment is derived from. For example, a biologically active fragment may be less than ten residues if the parent peptide is ten amino acid residues. Such peptides can be prepared by conventional solid phase synthesis techniques (SPPS) or expression techniques. In an example, a biologically active fragment or analog acts as a contact inhibitor for cancer (e.g., has anti-tumor properties) in mammals.

Examples of analogs include, but are not limited to function-conservative variants. “Function-conservative variants” are those in which a given amino acid residue in a protein/peptide has been changed without altering the overall conformation and/or function of the polypeptide, including, but not limited to, replacement of an amino acid with one having similar properties (e.g., polarity, hydrogen bonding potential, acidity, basicity, hydrophobicity, aromaticity, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine, or valine.

Various amino acids may be substituted with other amino acids having similar functionality. For example, serine may be replaced with threonine. Such changes are expected to have little or no effect on the apparent molecular weight or isoelectric point of the peptide. Amino acids other than those indicated as conserved may differ in a protein/peptide so the percent protein/peptide or amino acid sequence similarity between any two proteins/peptides of similar function may vary and may be, for example, from 70% to 99% as determined according to an alignment scheme, such as, for example, by the Cluster Method, where similarity is based on the MEGALIGN algorithm. A “function-conservative variant” also includes a polypeptide that has at least 60% amino acid identity as determined by BLAST or FASTA algorithms, preferably at least 90%, and which has the same or substantially similar properties or functions as the native or parent protein/peptide to which it is compared.

In an example, the purity of the cyclic peptide dimer or dimer comprising biologically active fragment/analog is at least 80%, at least 85%, at least 90%, at least 95%, at least 96%, at least 97%, at least 98%, at least 99%, or at least 99.9%. In various examples, the cyclic peptide dimer or biologically active fragment/analog is 100% pure.

In an aspect, the present disclosure provides pharmaceutical compositions comprising cyclic peptide dimers, biologically active fragments of the peptide(s) of the cyclic peptide dimers, and/or analogs thereof, and the like.

Various pharmaceutically acceptable carriers are suitable for compositions of the present disclosure. Pharmaceutically acceptable carriers may be determined in part by the particular composition being administered, as well as by the particular method used to administer the composition. Accordingly, there are a wide variety of suitable formulations of pharmaceutical compositions of the present disclosure. The vesicles may be freely suspended in a pharmaceutically acceptable carrier. Examples of carriers include solutions, suspensions, emulsions, solid injectable compositions that are dissolved or suspended in a solvent before use, and the like. The injections may be prepared by dissolving, suspending or emulsifying one or more of the active ingredients in a diluent. Examples of diluents, include, but are not limited to distilled water for injection, physiological saline, vegetable oil, alcohol, dimethyl sulfoxide, and a combination thereof. Further, the injections may contain stabilizers, solubilizers, suspending agents, emulsifiers, soothing agents, buffers, preservatives, etc. The injections may be sterilized in the final formulation step or prepared by sterile procedure. The composition of the disclosure may also be formulated into a sterile solid preparation, for example, by freeze-drying, and can be used after sterilized or dissolved in sterile injectable water or other sterile diluent(s) immediately before use. Additional examples of pharmaceutically acceptable carriers include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2005) 21st Edition, Philadelphia, Pa. Lippincott Williams & Wilkins. Effective formulations include, but are not limited to, oral and nasal formulations, formulations for parenteral administration, and compositions formulated for with extended release. Parenteral administration includes infusions such as, for example, intramuscular, intravenous, intraarterial, intraperitoneal, subcutaneous administration, and the like.

Examples of compositions include, but are not limited to, liquid solutions, such as, for example, an effective amount of a compound of the present disclosure suspended in diluents, such as, for example, water, saline or PEG 400. The liquid solutions described above may be sterile solutions. The compositions may comprise, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

The compositions may be presented in unit-dose or multi-dose containers, for example, sealed ampules and vials, and may be stored in a freeze-dried (lyophilized) condition requiring only the addition of the sterile liquid carrier, for example, water for injections, immediately prior to use (e.g., a kit). Extemporaneous injection solutions and suspensions may be prepared from sterile powders, granules and tablets of the kind previously described. The pharmaceutical compositions may also contain pharmaceutically acceptable carriers or diluents.

In various examples, a composition of the present disclosure comprises various combinations of different isomers (e.g., isoforms) of a cyclic peptide dimer. For example, a composition comprising two different isomers has various ratios of the different isomers (e.g., isoforms), such as, for example, 1:1 to 1:100 or 100:1, including every ratio value and range therebetween, or any possible ratio of the two isomers. In an example, a composition comprises or consists essentially of A and B, A and C, or B and C, where the ratio of A and B, A and C, or B and C is 1 to 1:100 or 100:1, including every ratio value and range therebetween, or any other ratio of A and B, A and C, or B and C. If there are three different isomers, the ratios of isomers may be 1:1:1 to 1:100:1, 1:1:100, 1:100:100, 100:1:100, or 100:100:1, including every ratio value and range therebetween, or any other ratio of the three isomers. In an example, a composition comprises or consists essentially of peptides A, B, and C, where the ratio of A, B, and C is 1:1:1 to 1:100:1, 1:1:100, 1:100:100, 100:1:100, or 100:100:1, including every ratio value and range therebetween, or any other ratio of A, B, and C. In various embodiments, the compositions do not contain any isomers other than A, B and/or C.

In various examples, a composition of the present disclosure comprises various combinations of different cyclic peptide dimers (e.g., different cyclic peptide dimers that are not isomers). For example, a composition comprising two different cyclic peptide dimers has various ratios of the different cyclic peptide dimers, such as, for example, 1:1 to 1:100 or 100:1, including every ratio value and range therebetween, or any possible ratio of the two different cyclic peptide dimers. If there are three different cyclic peptide dimers, the ratios of different cyclic peptide dimers may be 1:1:1 to 1:100:1, 1:1:100, 1:100:100, 100:1:100, or 100:100:1, including every ratio value and range therebetween, or any possible ratio of the three different cyclic peptide dimers.

In an example, the content of the cyclic peptide dimer, composition thereof, or dimer comprising a biologically active fragment/analog is 20% or less of a cyclic and/or a linear monomeric peptide (e.g., non-dimerized cyclic peptide and/or non-dimerized linear peptide), 15% or less of a cyclic and/or a linear monomeric peptide (e.g., non-dimerized cyclic peptide and/or non-dimerized linear peptide), 10% or less of a cyclic and/or a linear monomeric peptide (e.g., non-dimerized cyclic peptide and/or non-dimerized linear peptide), 5% or less of a cyclic and/or a linear monomeric peptide (e.g., non-dimerized cyclic peptide and/or non-dimerized linear peptide), 4% or less of a cyclic and/or a linear monomeric peptide (e.g., non-dimerized cyclic peptide and/or non-dimerized linear peptide), 3% or less of a cyclic and/or a linear monomeric peptide (e.g., non-dimerized cyclic peptide and/or non-dimerized linear peptide), 2% or less of a cyclic and/or a linear monomeric peptide (e.g., non-dimerized cyclic peptide and/or non-dimerized linear peptide), 1% or less of a cyclic and/or a linear monomeric peptide (e.g., non-dimerized cyclic peptide and/or non-dimerized linear peptide), or less than 1% of a cyclic and/or a linear monomeric peptide (e.g., non-dimerized cyclic peptide and/or non-dimerized linear peptide). In various examples, there is no detectable a cyclic and/or a linear monomeric peptide (e.g., non-dimerized cyclic peptide and/or non-dimerized linear peptide).

The compositions may be administered systemically. Compositions may be administered orally, may be preferably administered parenterally, and/or may be most preferably intravenously. Compositions suitable for parenteral, administration may include aqueous and/or non-aqueous carriers and diluents, such as, for example, sterile injection solutions. Sterile injection solutions may contain anti-oxidants, buffers, bacteriostatic agents and solutes, which render the composition isotonic with the blood of the intended recipient. Aqueous and/or non-aqueous sterile suspensions may include suspending agents and thickening agents.

Nasal aerosol and inhalation compositions of the present disclosure may be prepared by any method in the art. Such compositions may include dosing vehicles, such as, for example, saline; preservatives, such as, for example, benzyl alcohol; absorption promoters to enhance bioavailability; fluorocarbons used in the delivery systems (e.g., nebulizers and the like; solubilizing agents; dispersing agents; or a combination thereof).

The compositions of the present invention may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration. Preferably, the compositions are administered orally, intraperitoneally, or intravenously.

In an aspect, the present disclosure provides methods to inhibit the growth of malignant cells (e.g., methods for the treatment of tumors) in mammals. The methods of the present disclosure may be particularly suitable for solid tumors.

In various examples, the method comprises administering to a mammal in need of such treatment (e.g., a subject in need of treatment) a therapeutically effective amount of a composition comprising cyclic peptide dimers, including biologically active fragments of peptides, and/or analogs thereof effective to inhibit the growth and production of malignant cells (e.g., the malignant cells of one or more tumors) (e.g., treat a subject having one or more tumors).

The phrase “therapeutically effective amount” is used herein to mean an amount sufficient to reduce by at least about 15 percent, preferably by at least 50 percent, more preferably by at least 90 percent, and most preferably prevents growth and/or metastasis of a tumor and a clinically significant deficit in the activity, function and response of the host. Alternatively, a therapeutically effective amount is sufficient to cause an improvement in a clinically significant condition in the host (e.g., a reduction in the tumor burden).

Various solid tumors may be treated using methods of the present disclosure. Non-limiting examples of solid tumors that may be treated include sarcomas and carcinomas such as, for example: fibrosarcoma, myxosarcoma, chondrosarcoma, osteogenic sarcoma, angiosarcoma, endotheliosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, sweat gland carcinoma, sebaceous gland carcinoma, renal cell carcinoma, hepatoma, bile duct carcinoma, cervical cancer, testicular tumor, lung carcinoma, bladder carcinoma, epithelial carcinoma, melanoma, retinoblastoma, and the like, and combinations thereof.

Without intending to be bound by any particular theory, it is considered that the cyclic peptide dimers of the present disclosure act to reestablish in the cancer cells the contact inhibition of growth. Contact inhibition can prevent the unregulated reproduction of cells, such as, for example, malignant cells in tumors.

In an example, compositions comprising peptides A, B, and/or C are suitable contact inhibitors. Compositions comprising peptides D, E, and/or F are not effective contact inhibitors. Without intending to be bound by any particular theory, it is considered that acetamidomethyl (ACM) protecting groups of Cys residues of the cyclic peptides of the cyclic peptide dimers may decrease the efficacy of contact inhibition.

The steps of the method described in the various examples disclosed herein are sufficient to carry out the methods of the present disclosure. Thus, in an example, the method consists essentially of or consists of a combination of the steps of the methods disclosed herein.

The following example is presented to illustrate the present disclosure. It is not intended to be limiting in any matter.

### EXAMPLE 1

This example provides a description of synthesis and use of cyclic peptide dimers of the present disclosure.

Contact Inhibitory Factor is a Structurally Novel Endogenous Cyclic Decapeptide Dimer. Determining the Structure of the CIF Molecule—Overall Approach. Identifying the molecule responsible for restoring contact inhibition of growth to melanoma cells first required production of the CIF positive culture medium from the serum free AM cell line (SFAMCM), followed by column chromatography, electrophoresis and mass spectroscopy of this culture medium. Samples obtained after each procedure were evaluated for CIF activity with an in vitro bioassay (Bioassay described herein). Identification of CIF structure evolved in three stages: (1) the CIF active conditioned medium from the AM cells (SFAMCM) contained a single decapeptide; (2) the decapeptide is head-to-tail cyclic; (3) the complete bioactive CIF molecule is a dimer composed of identical disulfide linked cyclic decapeptides. An in vitro bioassay used to monitor and guide purification involved the contact inhibitory response of B16F10 mouse melanoma cells to test samples. The latter included (1) CIF-containing medium from both the AM and revertant FF cell lines; (2) all fractions derived from the AM medium during purification; (3) the synthetic CIF peptide. Results were evaluated independently by two investigators. Samples were graded from 0 to 4, where 0 indicated no activity and 4 indicated maximum activity. Only samples graded 3 or 4 by both investigators were selected for purification.

Purification: Isolation, Molecular Weight, Amino Acid Composition, Cyclic and Dimeric Structure. The starting material for purification was SFAMCM. Purification was done in two phases. In the first phase, a Waters MCX cartridge was used. In the second phase, HPLC with a Waters reverse phase column was used. The first (MCX) phase produced four fractions, H₂O, propanol (Prop), acetonitrile (ACN), and tetrahydrofuran (THF), of which three, Prop, THF, and ACN were positive in the bioassay. All four fractions were then further analyzed by HPLC. Only the resulting Prop fraction had activity on bioassay. The four fractions were then further analyzed by gel electrophoresis. Only the CIF positive Prop fraction stained (FIG. 1).

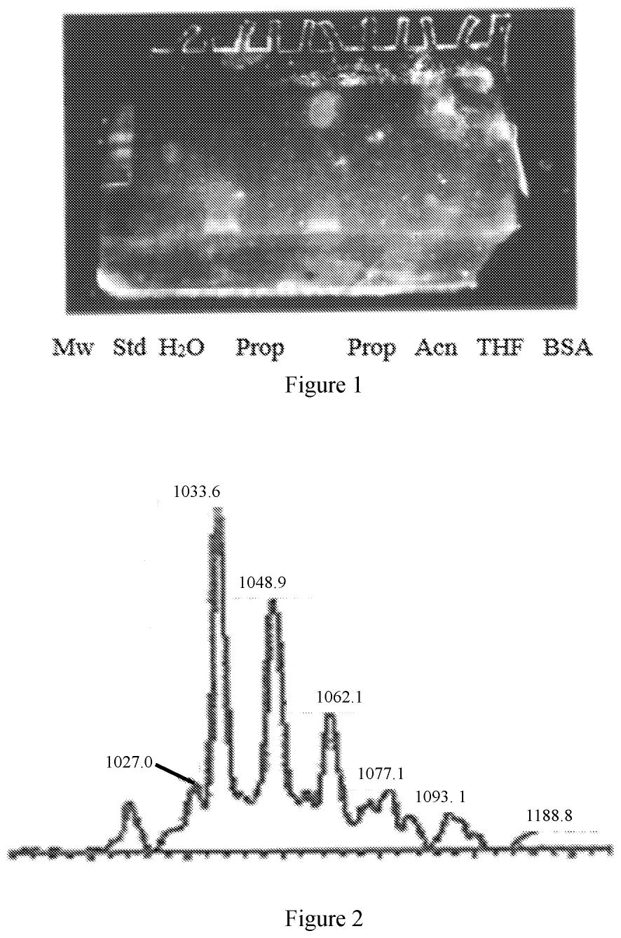

FIG. 1 shows gel electrophoresis of the fractions derived under reducing conditions from the HPLC separation of the MCX cartridge eluates. This revealed only a single CIF bioassay positive fraction, propanol. The H₂O, ACN, Prop and THF fractions from the HPLC were bioassayed. Only the Prop fraction had CIF activity. All four fractions were then electrophoresed using a 10-20% polyacrylamide gradient and stained with SYPRO. The running buffer contained the reducing agent mercaptoethanol. Only the Prop fraction stained revealing a single band; the other fractions, H₂O, ACN, and THF, did not stain indicating the absence of CIF. Only the Prop fraction was also bioassay positive for CIF activity, indicating the bioactive molecule was purified. This band was excised from the gel and analyzed by mass spectroscopy to determine molecular weight and amino acid composition and sequence of CIF. Mass spectroscopy showed that the apparent molecular weight of bioactive CIF obtained by electrophoresis under reducing conditions is 1033.5, with 4 additional peaks at 16 Da intervals indicating the capacity of CIF to bind up to 4 oxygen molecules (FIG. 2).

FIG. 2 shows the apparent molecular weight of CIF. Mass Spectrogram from electrophoresis of the single band of the CIF positive pooled propanol fractions derived from SFAMCM. This pooled fraction was the only one positive for CIF activity and had a single band on the electrophoretic gel, indicating a pure substance. The mass spectrogram had peaks at 1033.5, 1048.1, 1062.1, 1077.1 and 1093.1. The molecular weight differences are approximately 16, indicating the addition of 0-4 oxygens. The apparent molecular weight of CIF, the unoxygenated molecule, is approximately 1033.5.

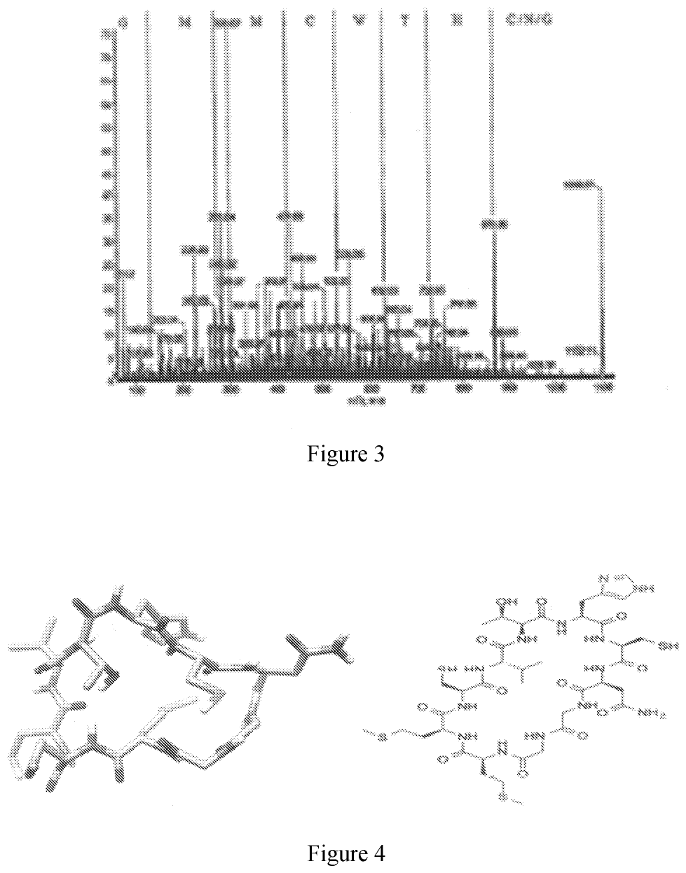

To determine the amino acid sequence of the peptide isolated from the single band of the propanol fraction derived from electrophoresis of the CIF positive HPLC fraction, we performed mass spectroscopy (FIG. 3).

FIG. 3 shows the amino acid composition and sequence of CIF peptide. Data obtained from the single band of the propanol fraction derived from electrophoresis of the CIF bioassay positive HPLC fraction showed a linear decapeptide whose amino acid composition and sequence is: Gly-Met-Met-Cys-Val-Thr-His-Cys-Asn-Gly (SEQ ID NO:1).

However, further analysis of these data led to the discovery that the CIF molecule is not linear, but actually cyclic. The mass spectrogram from the single band obtained by electrophoresis of the CIF positive propanol fraction from the SFAMCM had revealed that the apparent molecular weight of the unoxygenated Prop fraction of CIF, is 1033.5 Da. However, the calculated sum of the molecular weights of the amino acids is actually 1052 Da. The difference is 18 Da, the molecular weight of water. A molecule of water has been removed; therefore, the decapeptide has 10 amide bonds rather than 9. The additional amide bond is the result of head-to-tail cyclization of the linear molecule. CIF is a head-to-tail cyclic decapeptide (FIG. 4).

FIG. 4 shows the cyclic structure of the CIF molecule. The difference between the calculated MW of the CIF peptide (1052 Da) and its actual MW by mass spectrometry (1034.5 Da) is ˜18 Da, the MW of H₂O. A molecule of H₂O has been removed from the linear peptide to produce an additional amide bond resulting in a head-to-tail cyclic peptide.

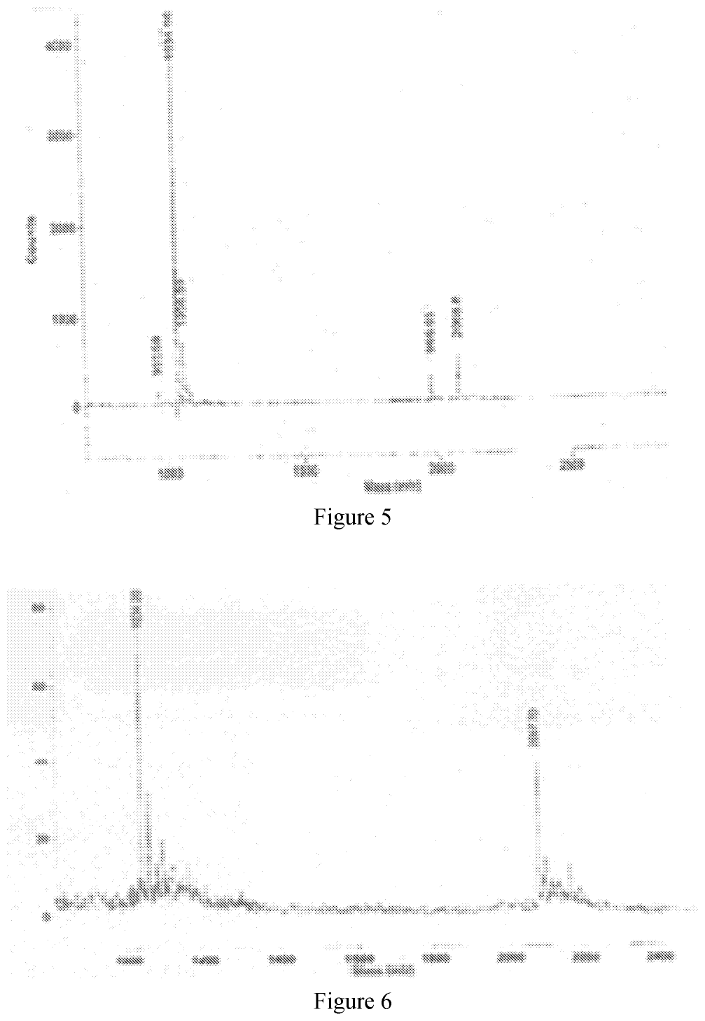

Having disclosed the cyclic nature of CIF, the cyclic molecule synthesized (Synthesis described herein) and further analyzed by mass spectroscopy (FIG. 5).

FIG. 5 indicates the existence of a CIF dimer. Mass spectroscopy of the synthetic CIF cyclic peptide revealed a large peak at 1034.66 Da, the expected molecular weight of cyclic CIF, but also a small peak at 2066.8 Da. The molecular weight of the small peak is approximately two times 1034.66, suggesting for the first time that CIF can exist as a dimer.

Thus, the CIF dimer was synthesized. (FIG. 6). FIG. 6 shows a mass spectrogram of the synthesized dimer. The resulting preparation was a mixture of the dimer and the monomer because much of the monomer remained undimerized. It is the peak at 1034 Da. The dimer is the peak at 2068 Da. The synthesized monomer and dimer were then separated by RP-HPLC and each separately bioassayed. The pure synthetic monomer was CIF negative; the synthetic dimer was CIF positive. The biologically active CIF molecule is a dimer consisting of two identical cyclic monomers.

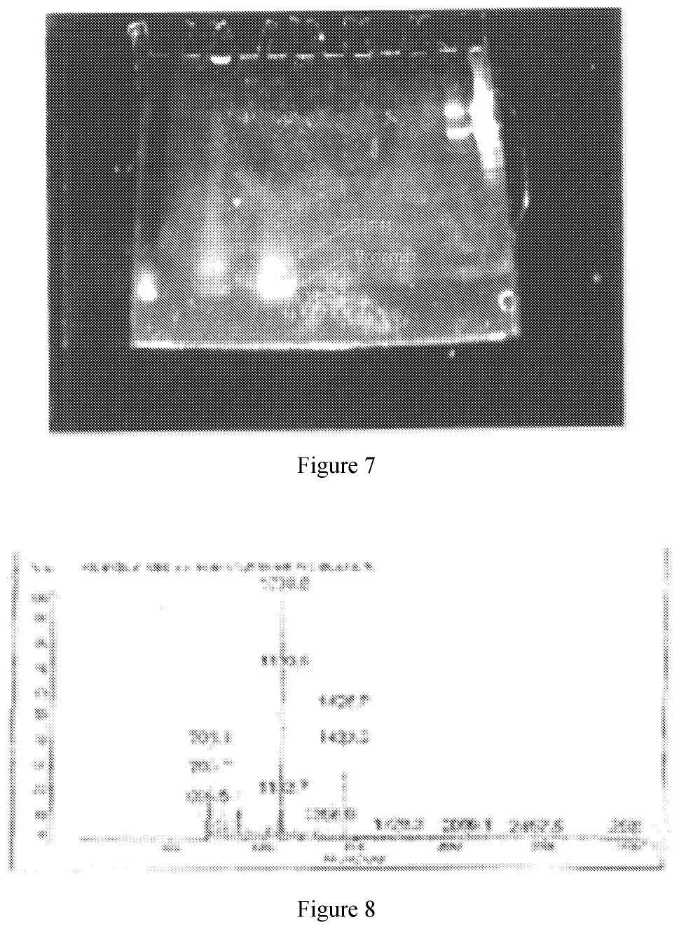

The only way a dimer can form from the CIF monomer is via disulfide bonds between one or both cysteines on each monomer. Because initial purification was completed under reducing conditions using β-mercaptoethanol, any disulfide bonds would have been split, leaving only monomers. When purification is repeated under non-reducing conditions, gel electrophoresis of the CIF active propanol fraction revealed that CIF exists as both dimer and monomer (FIG. 7).

FIG. 7 shows electrophoresis of the bioassay positive propanol fraction under non-reducing conditions. The procedure was performed as described in the section on electrophoresis (FIG. 1), omitting the mercaptoethanol. The non-reduced gel demonstrates that CIF exists as both a monomer and a dimer.

Discussion. There is evidence for plasticity of the malignant phenotype. Even with multiple genetic and epigenetic abnormalities cancer cells in specific tissue environments are capable of a wide range of bidirectional phenotypic variations ranging from highly malignant to benign. For example, environmentally mediated shifts toward a benign phenotype have been reported for such diverse malignancies as cancers of breast, prostate, colon and skin. The challenge has been to identify specific molecules which can translate the promise implicit in tumor cell plasticity to the clinical arena so as to shift the biologic balance in favor of host control.

Thus, a novel cyclic decapeptide dimer, CIF, derived from melanoma cells, reverses the melanoma malignant phenotype, restoring contact inhibition of growth, a key indicator of in vitro growth control which is correlated with normal cellular behavior in vivo.

All of these normalizing changes associated with restoration of contact inhibition were not only correlated and concurrent with, but also without exception contingent upon ability of the employed SFCM or derivatives thereof to restore contact inhibition of growth in an in vitro bioassay.

The fact that CIF-like activity on melanoma has also been detected in cell culture media of both human keratinocytes and normal melanocytes, together with the contact inhibitory effects of SFCM against tumor cells of such diverse histogenetic origin, indicates that CIF may be the ligand for a yet to be identified contact inhibitory checkpoint, one which is defective in cancer cells but can be pharmacologically corrected by CIF. Correction of this defect may both support and simplify treatment of the most intractable cancers by controlling replication at a single locus, while simultaneously triggering a potentially sustainable reversal of the malignant phenotype.

That this essential growth regulatory function is mediated by a head-to-tail cyclic peptide is surprising. Cyclic peptides are rare (only about 100 identified in all of nature) and of very ancient origin, being present in bacteria, fungi, plants, marine organisms and some animals. It is believed no cyclic peptide has previously been found to participate in regulation of cell proliferation.

Identification of the present molecules capable of restoring contact inhibited growth to a wide array of cancers and its obligatory correlation in melanoma cells with restoration of multiple other essential aspects of normal cell behavior, supports the possibility of an alternative approach to the treatment of currently intractable cancers.

Methods. Cell Lines. To purify the molecule responsible for restoring contact inhibition of growth we utilized three cell lines: (1) the contact inhibited FF cell line (American Type Culture Collection, ATCC, CRL—1479) (ATCC Manassas, Va.) whose CIF containing serum free conditioned medium (SFFFCM) served as positive control in the bioassays, (2) the closely related hamster amelanotic melanoma cell line (AM) (American Type Culture Collection, ATCC, CCL-49) (ATCC Manassas, Va.), which gave rise to the FF cell line, to produce serum free conditioned medium (SFAMCM) for purification studies, (3) B16F10 mouse melanoma cells (American Type Culture Collection, ATCC, CRL—6475) (ATCC Manassas, Va.) for the bioassays. The presence of serum in the FF culture medium interfered with the purification procedure because the CIF bound tightly to the serum proteins and attempts to remove CIF from the serum proteins didn't provide sufficient material to work with. Also, removal of serum from FF culture medium led to detachment of the cells and the detached cells did not produce CIF. However, AM cells remained attached in serum free medium and continued to produce CIF. Therefore the AM cell were selected, weaned to zero % serum to produce the serum free medium for purification.

Production of the CIF Positive Serum Free Culture Medium (SFFFCM) From FF Cells for Use as Positive Control. The culture medium for these studies was Dulbecco's Modified Eagle Medium (DMEM) with 10% FBS. One million FF cells were seeded into twelve 75 cm² flasks and incubated at 37° C. with 5% CO₂. The cells were refed every 2-3 days with fresh medium until confluent. The cells were then refed with serum free medium. After 2 days in SF medium, the medium was replaced with fresh serum free medium. The cells then were then incubated for 4-5 days at room temperature and the serum free medium collected. This was the material for the positive controls for the bioassays and for the studies which had demonstrated anti-angiogenesis, restoration of surface antigens, tumor inhibition and other effects as well. An aliquot was removed and bioassayed for CIF activity. The remaining amounts of the CIF positive samples were stored at 4° C. until 250-300 mls were accumulated. These were pooled, lyophilized and stored in desiccator jars under vacuum at 4° C.

Production of the CIF Positive Serum Free Culture Medium {SFAMCM) from AM Cells for Purification of the CIF Molecule. One million AM cells were seeded into 12 Primaria flasks at 37° C., 5% CO₂ in DMEM with 10% FBS. The cells were refed every 2-3 days with 20 mls of DMEM, 10% FBS until confluent. In order to remove the serum proteins that interfered with purification, the cells were weaned off serum by slowly reducing the serum concentration to 0%. The reduction was done at room temperature, 5% CO₂ to maintain the attachment of the cells. The reduction schedule was, 10%, 5%, 1%, 0.5%, 0.2%, 0.1% and 0% (serum free) and done daily. The cells can be left at any of these concentrations for a few days or over the weekend. After the first change to 0%—serum free medium—the medium was discarded and replaced with fresh serum free medium. The cells were again incubated for 3-4 days at 37° C., 5% CO₂ and the serum free medium collected. This is the AMCM and is the starting material for the purification. The cells slowly detached at 37° C. but they still produced enough SFAMCM to use as the starting material.

Bioassay. MCX Cartridge. The eluting solvents were HPLC grade H₂O, acetonitrile (ACN), 2-propanol (Prop) and tetrahydrofuran (THF). The organic solvents contained 0.1% trifluoroacetic acid (TFA). The serum free AMCM was reconstituted in 150 mls of dH₂O. 4.5 mls were removed, kept at 4° C. and reserved for the bioassay. The cartridge was equilibrated with PBS. All samples and solvents applied to the MCX cartridge were drawn through under vacuum. The cartridge was equilibrated with PBS. The remaining 145 mls of AMCM were loaded onto the cartridge, drawn through and collected. This material was consistently negative in the bioassay; therefore, subsequent collections were discarded. Then 185 mls of H₂O were run through the cartridge. This eluate was collected and designated the ‘H₂O₁’ fraction. The cartridge was then eluted with 35 mls of ACN-TFA. This eluate was designated ‘ACN₁’. The cartridge was then eluted with 35 mls of Prop-TFA, the eluate designated ‘Prop₁’. The cartridge was next eluted with 35 mls of THF-TFA, this eluate was designated ‘THF’. The cartridge was then eluted with 35 mls of Prop-TFA, this eluate designated ‘Prop₂’. The cartridge was then eluted with 35 mls of ACN-TFA, this eluate designated ‘ACN₂’. Then 35 mls of H₂O containing 0.1% TFA was flowed through the cartridge, and designated the ‘H₂O₂’ fraction. The H₂O fractions, the ACN fractions and the Prop fractions were each pooled. The pooled H₂O fractions were lyophilized. The pooled ACN and Prop fractions and the THF fraction were flash evaporated to remove the solvents and then lyophilized. For each of the pooled fractions, the next step was to prepare one aliquot for the bioassay and another for application to the HPLC. To do this, each lyophilized sample was redissolved in 9 mls of HPLC grade H₂O. For the bioassay, 3 mls were placed in a 15 mls centrifuge tube and, for the HPLC, 6 mls were placed into another 15 ml centrifuge tube and then each was lyophilized. The bioassays were done immediately including the pre-cartridge aliquot of the AMCM. The ACN, Prop and THF fractions were positive in the bioassay, the H₂O fraction was negative. The 6 ml aliquots were reserved for application to the HPLC.

HPLC. The HPLC column was stored in ACN. To prepare for the run, the ACN was flushed out with H₂O/0.1% TFA and the column equilibrated with the same solvent. Each of the three CIF positive fractions from the MCX cartridge, ACN, Prop and THF were dissolved in 0.5 ml of H₂O/0.1% TFA and then combined and brought up to 2 mls. The entire 2 mls was injected onto the HPLC column and was eluted with the same solvent using a gradient scheme (Table 1).

The tubes for each solvent from the HPLC were pooled forming the H₂O, ACN, Prop and THF fractions. The H₂O fraction was transferred into an Erlenmeyer flask and lyophilized. The lyophilized powder was dissolved in 5 ml of H₂O and then transferred to a 15 ml centrifuge tube. This was aliquoted into ⅕ and ⅘ portions, lyophilized and stored at 4° C. The other fractions were transferred to Florence flasks and flash evaporated to remove the organic solvents leaving a small amount of water which was then transferred to a 15 ml centrifuge tube. The flasks were then rinsed with water, which was added to the 15 ml tube. Water was then added bringing the volume up to 5 ml. All the fractions were aliquoted into ⅕ and ⅘ portions and then lyophilized and stored at 4° C. The ⅕ aliquot was used for the bioassay; the ⅘ aliquot for gel electrophoresis.

Electrophoresis. The H₂O, ACN, Prop and THF fractions were bioassayed. Only the Prop fraction had CIF activity. The electrophoresis was run using a 10-20% polyacrylamide gradient gel. The sample buffer was prepared by adding 50 μl of 2-mercaptoethanol to 950 μl of Laemmli sample buffer. Twenty μl of sample buffer was added to the dry (lyophilized) samples and heated at 95° C. for 5 minutes. Fifteen μl were then loaded on the gel. The Low Molecular weight standards were run in lane one. BSA was run in lanes 11 and 12 as a positive control for the SYPRO stain. The running buffer was Tris-Tricine. The gel was run at 120 volts for 1 hour and then stained with SYPRO Ruby protein stain (FIG. 1) Only the Prop fraction stained; the other fractions, H₂O, ACN, and THF did not stain. Only the Prop fraction was positive for CIF activity and it had only a single band. The stained band was cut out from the gel and analyzed as described in the section on mass spectroscopy.

The first lane contained the Low Molecular Weight Standard. Lanes 4 & 7 contained aliquots of the CIF positive propanol fraction. Lanes 11 and 12 contained BSA. Lane 2 contained the H₂O fraction, lane 9 the ACN fraction and lane 10 the THF fraction. The H₂O, ACN, Prop and THF fractions were bioassayed. Only the Prop fraction had CIF activity. The electrophoresis was run using a 10-20% polyacrylamide gradient gel. The sample buffer was prepared by adding 50 μl of 2-mercaptoethanol to 950 μl Laemmli sample buffer. Twenty μl of sample buffer was added to the lyophilized samples and heated at 95° C. for 5 minutes. Fifteen μl were then loaded on the gel. The Low Molecular Weight Standards were run in lane one. BSA was run in lanes 11 and 12 as a positive control for the SYPRO stain. The running buffer was Tris-Tricine. The gel was run at 120 volts for 1 hour and then stained with SYPRO Ruby protein stain.

Mass spectroscopy is shown in mass spectroscopy FIGS. 8-12.

The following materials were used for cell culture, column chromatography and electrophoresis (Materials).

Cell culture: 96 well flat bottom tissues culture plates, low evaporation lid, Fisher Catalog #07-200-89 Corning Costar Flat Bottom Plates Catalog #3595. Dulbecco's Modified Eagle Medium, Mediatech, Catalog #MT10-017-CV. Fetal Bovine Serum, Heat inactivated, Atlanta Biologicals, Catalog #S1150H. Glutamine solution: Powder, Atlanta Biologicals, Catalog #R90010, 100 gm. 20×solution: 6 gm Glutamine/500 ml PBS, sterile filter and aliquot into 20 ml/tubes. Store at −20° C. Add one 20 ml tube to 500 ml DMEM.

Pen/Strep, BioWhittaker, 10K/10K, Catalog #17-602E. Phosphate buffered saline, 1×, w/o Ca and Mg, Mediatech Catalog #MT21-031-CV. Primaria Tissue Culture Flasks, LDP Catalog #353810 Falcon 75 mm², straight neck, vented cap. Tissue Culture Flasks, LDP Catalog #353136, Falcon 75 mm², canted neck, vented cap. Trypsin-EDTA , Sigma Catalog #59418C. Water, LC-MS Grade, Thermo Scientific Pierce Water, Catalog #85189.

Column Chromatography: 2-Mercaptoethanol, Sigma-Aldrich, Catalog #M3148. 2-Propanol, Fisher, HPLC grade, Catalog #A451-4. Acetonitrile, Fisher, HPLC grade, Catalog #A998-4. HPLC Column, Waters, Milford, Ma., Xbridge Prep Shield RP18 OBD Column, 10 μm, 19×250 mm, Catalog #186003995. L-Glutamine, Atlanta Biologicals, Catalog #BR90010. MCX Cartridge, Waters, Milford, Ma., Oasis MCO, 35 ml, 6 gm, LP Extraction Cartridge, Catalog #186000778. Water, LC-MS Grade, Thermo Scientific Pierce Water, Catalog #85189.

Electrophoresis: Bovine Serum Albumin, Sigma-Aldrich, Catalog #A7906. Kaleidoscope polypeptide standards, Bio-Rad, Catalog #161-0325. Laemmli Sample Buffer, 2×, Bio-Rad, Catalog #161-0737. Mini-PROTEAN Tris-Tricine Precast Gel, 10-20%, Bio-Rad, Catalog #456-3116. Tetrahydrofuran, Fisher, HPLC grade, Catalog #T-425-1. Trifluoroacetic acid, Fisher, 50 ml, Catalog #LC-MS 85183. Tris-Tricine/SDS Bio-Rad Catalog #161-0744. Trypan Blue, Lonza, 0.4% solution in 0.85% NaCl, Catalog #17-942E. SYPRO Ruby Protein Gel Stain, Bio-Rad, 1×, 1L, Catalog #170-3125.

TABLE 1
Elution schedule from the HPLC of the AMCM.

| | Time | Flow | Water | Acetonitrile | Propanol | THF |
|---|---|---|---|---|---|---|
| Step | (min) | (ml/min) | (%) | (%) | (%) | (%) |
| 1 | 0.01 | 11 | 100 | 0 | 0 | 0 |
| 2 | 7 | 11 | 100 | 0 | 0 | 0 |
| 3 | 12 | 11 | 0 | 100 | 0 | 0 |
| 4 | 24 | 11 | 0 | 100 | 0 | 0 |
| 5 | 36 | 11 | 0 | 0 | 100 | 0 |
| 6 | 41 | 11 | 0 | 0 | 100 | 0 |
| 7 | 48 | 11 | 0 | 0 | 0 | 100 |
| 8 | 55 | 11 | 0 | 0 | 0 | 100 |
| 9 | 64 | 11 | 0 | 0 | 100 | 0 |
| 10 | 73 | 11 | 0 | 0 | 100 | 0 |
| 11 | 80 | 11 | 0 | 100 | 0 | 0 |
| 12 | 83 | 11 | 0 | 100 | 0 | 0 |
| 13 | 90 | 11 | 100 | 0 | 0 | 0 |
| 14 | 119 | 11 | 100 | 0 | 0 | 0 |

The tubes were pooled according to the following scheme:

| Tube # | Fraction |
|---|---|
| 1-26 | H₂O₁ |
| 27-45 | Acn₁ |
| 46-60 | Prop₁ |
| 61-80 | Thf |
| 81-100 | Prop₂ |
| 101-113 | Acn₂ |
| 114-120 | H₂O₂ |

Phase 1 of the purification of the AMCM using an MCX cartridge. An aliquot of the lyophilized AMCM was passed through an MCX cartridge and four fractions were collected, H₂O, ACN, Prop and THF. An aliquot of each fraction was bioassayed. The ACN, Prop and THF were positive for CIF activity. They were pooled, lyophilized and applied to the HPLC. Again four fractions were collected H₂O, ACN, Prop and THF according to the elution schedule shown here. A portion of each fraction was bioassayed and the rest reserved for electrophoresis. Only the Propanol fraction was positive in the bioassay.

Bioassay. Preparation of Samples. Conditioned Culture Medium (SFFFCM) and (SFAMCM). The positive control was the CIF positive conditioned culture medium from the FF cell line, SFFFCM. The negative control was DMEM 10% FBS. 3.6 ml of the sample was pipetted into a sterile 15 ml tube and heat activated at 80° C.×10 mins (minutes). After cooling to room temperature 0.4 ml FBS was added to make the FBS concentration 10%. For different volumes of conditioned culture medium the appropriate volume of FBS was added to make the final FBS concentration 10%. Both SFFFCM and SFAMCM were lyophilized and stored in desiccator jars under vacuum at 3° C.

Lyophilized Samples. To prepare the SFFFCM and the SFAMCM for the bioassay they were reconstituted with DMEM, heat activated at 80° C.×10 mins and cooled to room temperature. Sufficient volume of FBS was added to make the final FBS concentration 10%. The final volume of the sample was at least 2 ml to ensure that there was enough material for refeeding the bioassay plates.

Dilution of Samples. The samples were tested at 1:1 (undiluted), 1:2 and 1:4. The dilutions were made with DMEM 10% FBS. Higher dilutions may be used as needed. Each well contained 0.2 ml of sample, including the positive and negative controls. Each sample was done in duplicate. The wells were refed with fresh sample after 48 hrs.

Setting up the Bioassay. Using a 96 well flat bottom plate, 0.2 ml sterile water was pipetted into all outside wells. 0.2 ml 10% DMEM containing 20,000 B16F10 cells were pipetted into each well for the bioassay. Each sample was done in duplicate, including positive and negative controls. The wells were refed with fresh sample after 48 hrs. The plate was placed in a 37° C., 5% CO₂ incubator for approximately 4 hours to allow for cells to attach and spread. The time for attachment may vary for other cell lines. After the cells had attached and spread, the DMEM was removed and replaced with 0.2 ml of sample, positive and negative controls. The wells were observed daily and scored for CIF activity. The wells were refed after 48 hours or as needed, 0.2 ml for weekdays, 0.3 ml for weekends.

Scoring for CIF Activity. The samples were scored for CIF activity as follows:

4+=All cells are contact inhibited, flattened with an elongated fibroblast-like morphology.
3+=Not all cells are contact inhibited, elongated or fibroblast-like. There are a few scattered clusters of dividing cells.
2+=Fewer contact Inhibited cells and more clusters of dividing cells.
1+=Many clusters of dividing cells, very few elongated non-dividing.
0=No morphological change and no inhibition of growth.

Mass Spectroscopy (Mass Spectroscopy FIGS. 8-12). To determine the molecular weight and amino acid sequence of the peptide isolated from the pooled propanol fractions after electrophoresis, we used the SFAMCM. The analysis required the use of a Kratos Axima MALDI-TOF Mass Spectrometer (MS) and an Applied BioSystems Qstar Pulsar XL MALDI-Q-TOF CID-MS/MS instrument due to the hydrophobicity of the peptide and the non-ionizable nature of the peptide by ESI. The Prop band was excised from the electrophoretic gel and the material was extracted and prepared for analysis by MALDI-TOF mass spectroscopy following the manufacturer's ZIP TIP protocol. The Prop fraction sample was eluted from the Zip Tip with 0.1% TFA, 80% ACN containing 2.00 mg/ml α-cyano-4-hydroxy -cinnamic acid and the eluate then placed directly onto the MALDI target and allowed to dry. MALDI spectra were first acquired on the Kratos Axima CFR MALDI TOF for molecular weight analysis. A duplicate procedure was then performed on the Prop fraction and analyzed on the AB Qstar XL equipped with a MALDI ionization source. This sample was prepared in the same way with the exception of the matrix solvent, α-cyano-4-hydroxycinnamic acid, which was dissolved in 70% 2-propanol, 0.1% TFA. It is the MALDI Matrix and we got better signal on the Qstar by dissolving it in propanol rather than 80% acetonitrile. Data acquired on the Qstar was subjected to tandem mass spectroscopy (MS/MS) to elucidate the peptide sequence.

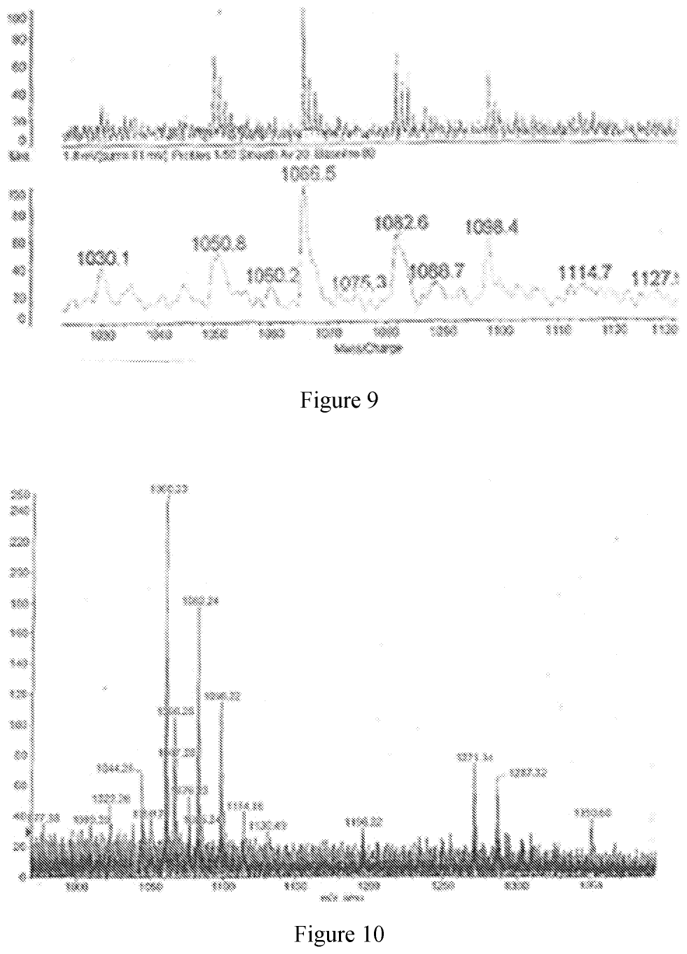

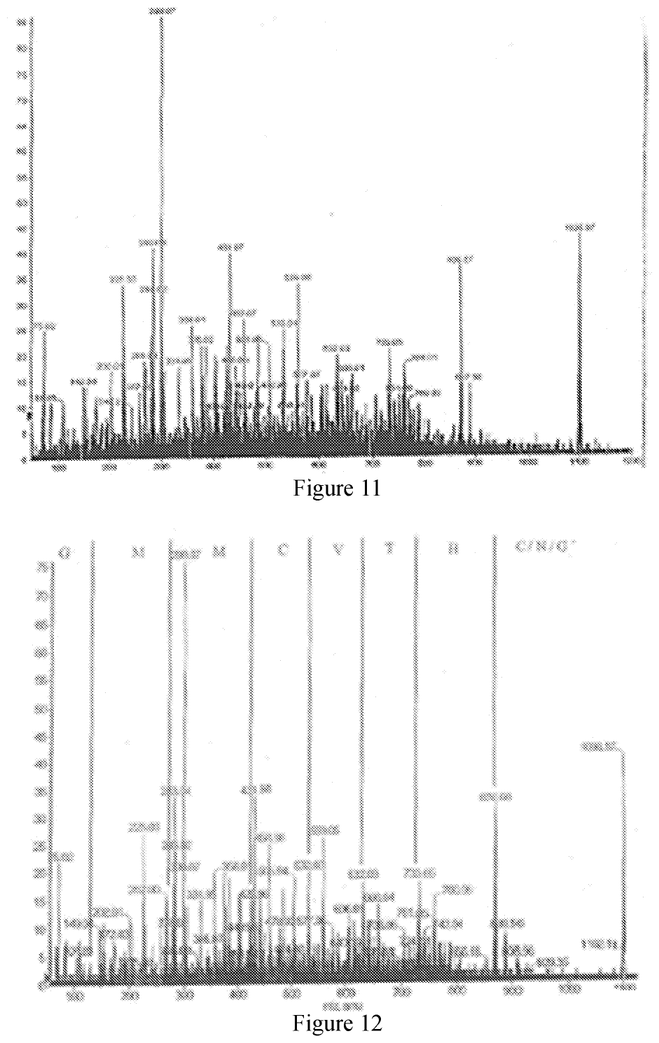

The three samples, Prop, ACN and THF, were evaluated on the Kratos MALDI-TOF MS instrument. The Prop fraction yielded two strong ions at 1428.9 and 1098.8 (FIG. 8). No other ions were seen in the ACN or THF fractions. Evaluation of the 1098.8 ion from the Prop fraction showed a series of ions at 1066, 1082 along with the 1098 ion (FIG. 9). The 16 AMU difference is due to the oxidation of methionine where the 16 additional mass units are from the addition of oxygen. The Prop fraction was further analyzed using the Qstar XL instrument with the MALDI source. The TOF-MS spectra clearly show the 1066, 1082 and 1098 methionine oxidation series along with 1060 as the main ions in the spectra (FIG. 10). MS/MS of the 1098 ion was performed generating a peptide fragment ion pattern (FIG. 11). De novo sequencing of the fragmentation ions was performed using Analyst QS software allowing for the identification of the amino acid sequence. The latter is seen in the annotated sequence of the 1098 MS/MS spectra (FIG. 12). The amino acid sequence is: Gly-Met-Met-Cys-Val-Thr-His-Cys-Asn-Gly (SEQ ID NO:1).

Synthesis. The CIF molecule was prepared by the solid state peptide syntheses method of Freeman in Goodman, Synthesis of Peptides and Peptidomimetics, Houben-Weyl, Stuttgart, 2002.

326573 c (Gly-Met (O)-Met (O)-Cys-Val-Thr-His-Cys-Asn-Gly (SEQ ID NO:1, where the methionine residues are oxidized)).

324822 c (Gly-Met-Met-Cys-Val-Thr-His-Cys-Asn-Gly (SEQ ID NO:1)) w/Cys-Cys bridge 326573 Resin: 2.4 g of Fmoc-Val-PEG Resin (sub.: 0.21 mmol/g).

Peptide Synthesis: The peptide was synthesized by Fmoc-chemistry starting with Val. Coupling condition: 3.3 equiv. of Fmoc-AA-OH and 3.3 equiv. of HBTU, HOBt and NMM. The coupling was monitored by the Ninhydrin Test.

Cleavage: The cleavage was done by Reagent K, 2.5 hr (hours) at RT. Yield 710 mg linear unprotected crude peptide.

Oxidation: 200 mg crude peptide was dissolved in 150 ml DMF and the pH was adjusted to 8 by DIPEA. The oxidation was completed after three days (monitored by MS).

Cyclization: The oxidized peptide solution was added to 50 ml DMF (containing 520 mg PyBop, 136 mg HOBt and 350 μl DIPEA) dropwise. The mixture was stirred at room temperature overnight and then concentrated to 5 ml after completion (checked by MS). The peptide was isolated by passing through RP-HPLC column (from Waters Corp.); the purity of the peptide was 99% on the profile.

Peptide 324822 was not the precursor of peptide 326573. It was synthesized separately. For 324822, protected linear peptide was cleaved from resin and cyclized in DMF following the above procedure. After removing the protecting groups, the peptide was isolated by RP-HPLC purification. Both peptides were not soluble in 100% aqueous solution. 10% acetonitrile in water was used to dissolve the peptides.

For 326573, was methionine sulfoxide synthesis using Fmoc-Met (O)—OH (methionine sulfoxide). Both Met are with sulfoxides. The peptide was identified by mass analysis.

The following is a key to the terms in the above protocol: HBTU—a coupling agent; 1-H-Benzotriazolium, 1-[Bis (dimethylamino) methylene]-hexafluorophosphate (1-), 3-oxide. NMM—another coupling agent; N-methylmorpholine. DMF—dimethyl formamide. DIPEA—N,N-diisopropylethylamine. PyBop—a peptide coupling agent; benzotriazol-1-yl-oxytripyrrolidinophosphonium hexafluorophosphate. RP-HPLC—reverse phase high performance liquid chromatography. HOBt—1-hydroxybenzotriazole hydrate.

Although the present disclosure has been described with respect to one or more particular embodiments and/or examples, it will be understood that other embodiments and/or examples of the present disclosure may be made without departing from the scope of the present disclosure.

1. A cyclic peptide dimer, comprising a first peptide comprising at least one Cys residue and a second peptide comprising at least one Cys residue, wherein the first peptide and the second peptide are connected via one or more bonds between the Cys residue of the first peptide and the Cys residue of the second peptide, and the first peptide and the second peptide are cyclic and the first peptide and the second peptide are 8 to 10 amino acid residues.

2. The cyclic peptide dimer of claim 1, wherein the first peptide and second peptide independently are chosen from Gly-Met-Met-Cys-Val-Thr-His-Cys-Asn-Gly (SEQ ID NO:1), Gly-Met-Met-Cys-Val-Ser-His-Cys-Asn-Gly (SEQ ID NO:2), Cys-Met-Met-Asn-Thr -Ser-Cys-Met-Val-Leu (SEQ ID NO:3), Cys-Met-Met-Asn-Thr-Ser-Cys-Met-Val-Ile (SEQ ID NO:4), and combinations thereof.

3. The cyclic peptide dimer of claim 1, wherein the first peptide and second peptide are connected with a second disulfide bond formed between a second Cys residue of the first peptide and a second Cys residue of the second peptide.

4. The cyclic peptide dimer of claim 2, wherein one or more of the methionine residues are oxidized and/or one or more of the amino acid residues are sidechain protected.

5. The cyclic peptide dimer of claim 4, wherein the Cys is trityl-protected, Thr is t-butyl-protected, His is trityl-protected, and/or Asn is trityl-protected.

6. The cyclic peptide dimer of claim 1, wherein the cyclic peptide dimer is at least 80% pure.

7. A composition comprising a cyclic peptide dimer of claim 1 and a pharmaceutically acceptable carrier.

8. The composition of claim 7, further comprising one or more additional cyclic peptide dimers, wherein the one or more additional cyclic peptide dimers is/are structurally different than the cyclic peptide dimer.

9. The composition of claim 8, wherein the one or more additional cyclic peptide dimers are isoforms of the cyclic peptide dimer.

10. The composition of claim 7, wherein the composition has 20% or less of a cyclic and/or a linear monomeric peptide.

11. The composition of claim 10, wherein the composition has less than 1% of a cyclic and/or a linear monomeric peptide.

12. A method for inhibiting growth of malignant cells in a mammal in need of treatment comprising administering a therapeutically effective amount of a composition of claim 7 to the mammal in need of treatment, wherein the growth of the malignant cells in the mammal in need of treatment is inhibited.

13. The method of claim 12, wherein one or more tumors comprises the malignant cells.

14. The method of claim 13, wherein the one or more tumors is/are a sarcoma and/or carcinoma.

15. The method of claim 14, wherein the tumor is chosen from fibrosarcoma, myxosarcoma, chondrosarcoma, osteogenic sarcoma, angiosarcoma, endotheliosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, sweat gland carcinoma, sebaceous gland carcinoma, renal cell carcinoma, hepatoma, bile duct carcinoma, cervical cancer, testicular tumor, lung carcinoma, bladder carcinoma, epithelial carcinoma, melanoma, retinoblastoma, and combinations thereof.
